(19) United States
(12) Patent Application Publication
(10) Pub. No.: US 2023/0364348 A1
(43) Pub. Date: Nov. 16, 2023

(54) Biasing Member for Medical Injection Device

(71) Applicant: Becton, Dickinson and Company, Franklin Lakes, NJ (US)

(72) Inventors: Bradley M. Wilkinson, North Haledon, NJ (US); Craig Schneider, Sparta, NJ (US); Xin Xie, Montville, NJ (US); Richard A. Cronenberg, Gladstone, NJ (US); John Paul Hoffman, Somerset, NJ (US); Michael Vincent Quinn, East Hanover, NJ (US)

(21) Appl. No.: 18/028,252

(22) PCT Filed: Sep. 23, 2021
(86) PCT No.: PCT/US2021/051735

§ 371 Date: Mar. 24, 2023

Related U.S. Application Data
(60) Provisional application No. 63/083,564, filed on Sep. 25, 2020.

(51) Int. Cl. A61M 5/315; A61M 5/24

(52) U.S. Cl. CPC A61M 5/31535; A61M 5/24; A61M 2005/2477

(57) ABSTRACT

A biasing member for a medical injection device having a housing, a brake member, a cartridge, and an injection needle, wherein the biasing member includes a body having a first end, a second end positioned opposite the first end, a top surface, and a bottom surface positioned opposite the top surface, and an extension portion extending from the body in a direction extending from the top surface of the body to the bottom surface of the body. The extension portion is resilient.

### CROSS-REFERENCE TO RELATED APPLICATION

The present application claims priority to U.S. Provisional Application Serial No. 63/083,564, entitled “Biasing Member for Medical Injection Device”, files Sep. 25, 2020, the entire disclosure of which is hereby incorporated by reference in its entirety.

### BACKGROUND OF THE INVENTION

### Field of the Invention

The present disclosure relates generally to a biasing member for a medical injection device.

### Description of Related Art

Medical injection devices often utilize resilient members, such as springs, placed between various components, to bias the components in order to ensure more accurate drug delivery. However, there are shortcomings to the use of current resilient members used in medical injection devices, such as a lack of control and load recovery. Accordingly, there is a need in the art for a more robust component to allow for greater load recovery.

### SUMMARY OF THE INVENTION

In one aspect or embodiment, a biasing member for a medical injection device having a housing, a brake member, a cartridge, and an injection needle, includes a body having a first end, a second end positioned opposite the first end, a top surface, and a bottom surface positioned opposite the top surface, an extension portion extending from the body in a direction extending from the top surface of the body to the bottom surface of the body, where the extension portion is resilient.

The extension portion may extend from the bottom surface of the body. The body may be arcuate. The extension portion may be wavy. The body may define a width extending from the first end of the body to the second end of the body, with the extension portion having a width less than the width of the body. The biasing member may be T-shaped. The extension portion may extend from the body at an equidistant point between the first and second ends of the body. A distal end of the extension portion may be non-coplanar with the body. The biasing member may be formed from a metallic material. The body may be integrally formed with the extension portion. The body and the extension portion may be equal in thickness.

In a further aspect or embodiment, a medical injection device includes a housing having a distal end and a proximal end, a brake member received within the housing, a cartridge received within the housing, the cartridge configured to receive a medicament, a cartridge housing received within the housing distally of the brake member and configured to hold the cartridge, a biasing member as described in any of the aspects or embodiments above, with the biasing member received within the housing and positioned between the brake member and the cartridge, an injection needle at a distal end of the housing and in fluid communication with the cartridge, and an actuation member configured to actuate the medical injection device to deliver the composition through the injection needle.

The biasing member may be received within a pocket defined by the brake member. The pocket may be defined by a radially extending protrusion of the brake member. The biasing member may be first and second biasing members.

In another aspect or embodiment, a medical injection device includes a housing having a distal end and a proximal end, a brake member received within the housing, a cartridge received within the housing, the cartridge configured to receive a medicament, a cartridge housing received within the housing distally of the brake member and configured to hold the cartridge, a biasing member received within the housing and positioned between the brake member and the cartridge, with the biasing member formed as a compression spring, an injection needle at a distal end of the housing and in fluid communication with the cartridge, and an actuation member configured to actuate the medical injection device to deliver the composition through the injection needle.

The biasing member may be received within a pocket defined by the brake member. The pocket may be defined by a shoulder portion of the brake member, with the shoulder portion extending radially outward from a body of the brake member. The shoulder portion may include a protrusion extending radially inward into the pocket, the protrusion engaging a portion of the biasing member to retain the biasing member within the pocket.

### BRIEF DESCRIPTION OF THE DRAWINGS

FIG. 1 is a perspective view of a medical injection device according to one aspect or embodiment of the present application.

FIG. 2 is an exploded view of the medical injection device of FIG. 1.

FIG. 3 is a partial cross-sectional view of the medical injection device of FIG. 1, showing first and second biasing members in a pre-engagement position.

FIG. 4 is a partial cross-sectional view of the medical injection device of FIG. 1, showing first and second biasing members in an engagement position.

FIG. 5 is a partial perspective view of a brake member and biasing members of the medical injection device of FIG. 1.

FIG. 6 is a perspective view of a first and second biasing member according to one aspect or embodiment of the present application.

FIG. 7 is a partial cross-sectional view of the medical injection device of FIG. 1 according to a second aspect or embodiment of the present application, showing a biasing member in a pre-engagement position.

FIG. 8 is a partial cross-sectional view of the medical injection device of FIG. 7, showing a biasing member in an engagement position.

FIG. 9 is a perspective view of the biasing member of the medical injection device of FIG. 7.

### DESCRIPTION OF THE INVENTION

The use of numerical values in the various ranges specified in this application, unless expressly indicated otherwise, are stated as approximations as though the minimum and maximum values within the stated ranges are both preceded by the word “about”. As used herein, the term “about” means the stated value ± 10%. In this manner, slight variations above and below the stated ranges can be used to achieve substantially the same results as values within the ranges. Also, unless indicated otherwise, the disclosure of these ranges is intended as a continuous range including every value between the minimum and maximum values. For definitions provided herein, those definitions refer to word forms, cognates and grammatical variants of those words or phrases.

The figures accompanying this application are representative in nature, and should not be construed as implying any particular scale or directionality, unless otherwise indicated. For purposes of the description hereinafter, the terms “upper”, “lower”, “right”, “left”, “vertical”, “horizontal”, “top”, “bottom”, “lateral”, “longitudinal” and derivatives thereof shall relate to the invention as it is oriented in the drawing figures. However, it is to be understood that the invention may assume various alternative variations and step sequences, except where expressly specified to the contrary. Hence, specific dimensions and other physical characteristics related to the embodiments disclosed herein are not to be considered as limiting.

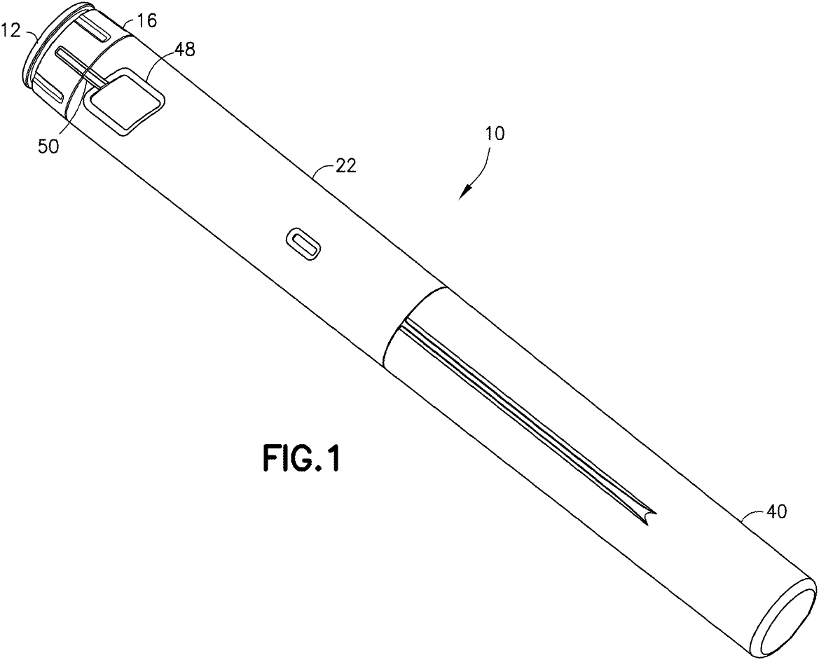

Referring to FIGS. 1-6, according to one aspect or embodiment of the present application, a medical injection device 10 includes a button 12, button spring 14, dose set knob 16, bearing insert 18, driver 20, housing 22, first and second biasing members 24, 26, brake member 28, lead screw 30, piston rod 32, stopper 34, cartridge 36, cartridge housing 38, cap 40, outer shield 42, and needle 44. The medical injection device 10 is utilized to deliver a medicament or composition contained within the cartridge 36 to a person. The medical injection device 10 may be similar and operate in the same manner as the injection device shown and described in U.S. Pat. No. 9,421,334, which is incorporated herein by reference in its entirety.

Referring to FIGS. 1-3, the housing 22 receives a plurality of dose setting and injection components. The housing 22 is connected to the cartridge housing 38, which receives the cartridge 36. The cap 40 covers the cartridge 36 and the cartridge housing 38 when the injection device 10 is not in use. The dose set knob 16 includes a knob-like portion that is rotated by a person to set a desired dose. The dose set knob 16 can also include a plurality of numerals or markings 46 that correspond to a number of dosage units that are visible through a window 48 provided on the housing 22. The dose set knob 16 is rotated until the desired dose is visible in the window 48. The housing 22 can include an arrow or other indicator 50 to precisely indicate the set dose. Once the desired dose is set, the button 12 is pressed until the set dosage amount is completely injected. The outer shield 42 can cover the needle 44 to prevent accidental needle sticks upon removal of cap 40.

The button 12 can include an annular bead or rim 52 that engages with a corresponding annular groove (not shown) provided on the internal surface of the dose set knob 16. The annular rim 52 and groove connection can be a friction fit that maintains the push button 12 in a biased position on the dose set knob 16 under the force of the button spring 14, but allows the button 12 to be pushed into the dose set knob 16 for injecting a set dose. The interior of the button 12 can accommodate the bearing insert 18 that rests on an internal surface at a proximal end of the driver 20. The push button 12 can be designed to rotate freely on the bearing insert 18.

The driver 20 can be a cylindrical member, coaxial with and surrounded by the dose set knob 16. The driver 20 can be provided co-axially around the brake member 28, which is axially and rotatably fixed to the housing 22. The brake member 28 co-axially surrounds the piston rod 32. The piston rod 32 includes a set of keys (not shown) that engage a slot (not shown) internal to the brake member 28 to rotatably lock the piston rod 32 to the brake member 28. The piston rod 32 can include a plurality of threads (not shown) provided on the interior surface thereof. The piston rod 32 co-axially surrounds the lead screw 30, which includes a series of threads 54 at least at its distal end. The lead screw threads 54 are configured to be in threaded engagement with the internal threads (not shown) provided on the interior of the piston rod 32. Due to its threaded engagement with the lead screw 30, the piston rod 32 can be moved into the cartridge 36 during injection to press on the stopper 34 provided inside the cartridge 36 to expel a dose of medication from the cartridge 36. To set a dose using the injection device 10 as described herein, a user rotates the knob portion of the dose set knob 16 relative to the housing 22. An outer surface of the dose set knob 16 can include a thread 56 that is in threaded engagement with a plurality of threads 58 provided on the internal surface of the housing 22. Accordingly, as the dose set knob 16 is rotated relative to the housing 22, the dose set knob 16 screws or advances a distance out of the housing 22.

With regard to the injection mechanism, as described above, the lead screw 30 includes the plurality of threads 54 at its distal end that engage with a plurality of threads (not shown) provided along the entire length of a hollow piston rod 32. The piston rod 32 is held non-rotatably with respect to the housing 22 due to a non-rotatable coupling with the brake member 28, which can be held axially and rotatably fixed with respect to the housing 22. The piston rod 32 can include a key or set of keys 60 at its distal end that engage with a slot (not shown) provided on the internal surface of the brake member 28 to prevent relative rotation therebetween while permitting the piston rod 32 to move axially with respect thereto. The threads 54 of the lead screw 30 can include a flat portion (not shown) corresponding to a flat portion 62 of the piston rod 32, such that axial movement of the lead screw 30 during dose setting does not result in axial movement of the piston rod 32. Accordingly, rotation of the lead screw 30 during injection of a dose can cause the threads 54 of the lead screw 30 to engage the threads of the piston rod 32, thereby axially moving the piston rod 32. Because the piston rod 32 can be non-rotatable with respect to the housing 22, as the lead screw 30 is caused to rotate during injection, as described above due to its rotational coupling with the driver 20, the piston rod 32 through its threaded engagement with the lead screw 30 can be caused to move in the distal direction to press against the stopper 34 provided in the medicament cartridge 36, thus expelling a liquid medication therefrom.

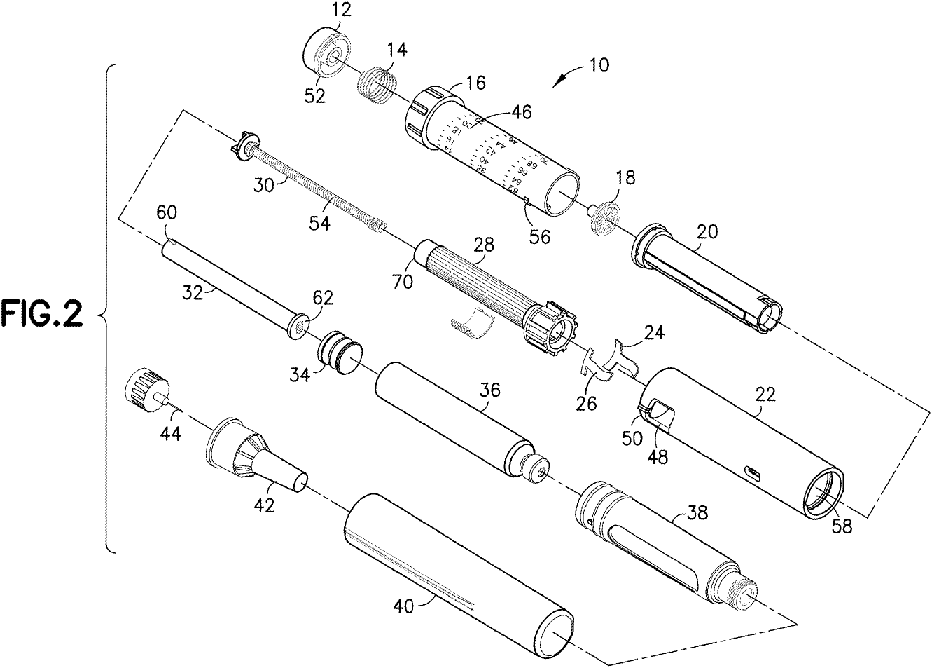

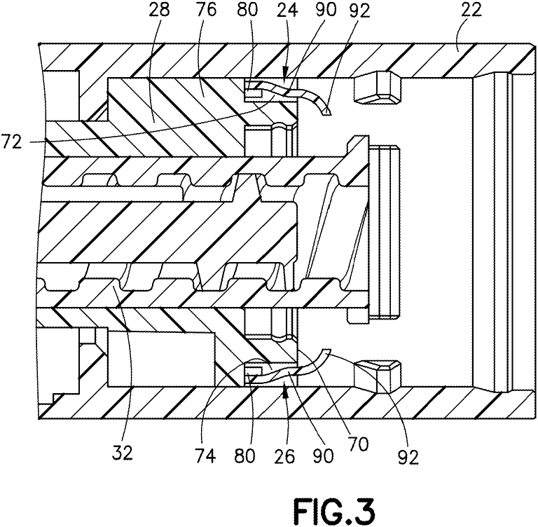

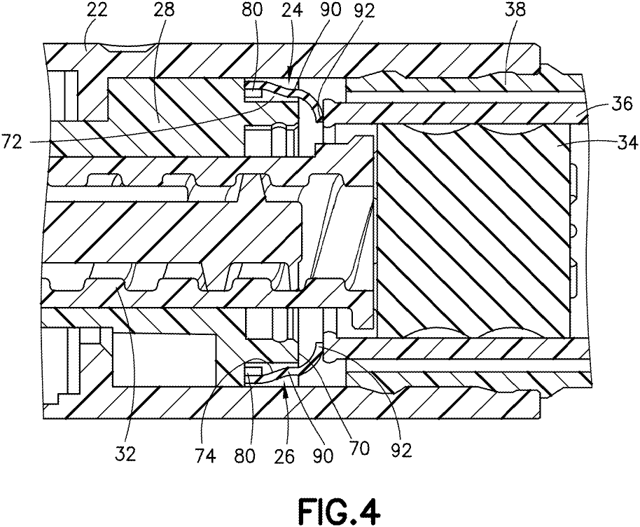

Referring to FIGS. 2-6, the first and second biasing members 24, 26 of the injection device 10 are positioned between a distal end 70 of the brake member 28 and the cartridge 36 in an axial direction to bias the cartridge 36 in a distal direction to prevent movement of the cartridge 36 during injection thereby ensuring an accurate dose is injected. As shown in FIGS. 3 and 4, during assembly of the device 10 and prior to insertion of the cartridge 36 (FIG. 3), the biasing members 24, 26 have an unbiased position where the biasing members 24, 26 have a maximum length. After assembly of the device 10 with the cartridge 36 inserted and engaged with the biasing members 24, 26 (FIG. 4), the biasing members 24, 26 are in a biased position where the biasing members 24, 26 are compressed with a length less than the maximum length of the biasing members 24, 26.

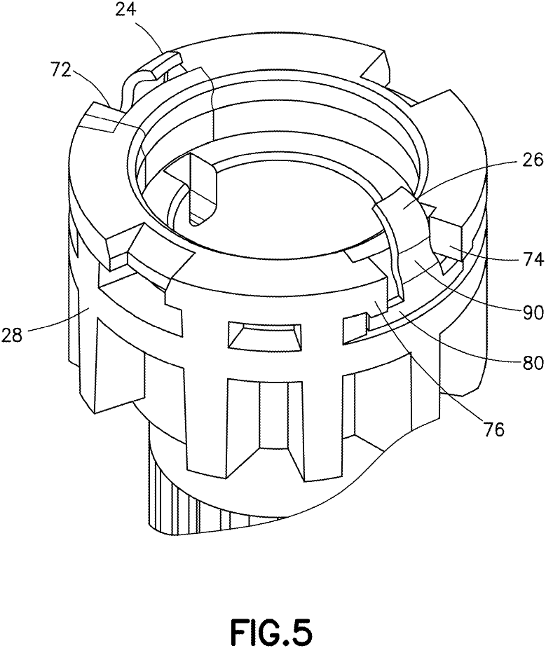

The first and second biasing members 24, 26 are also sandwiched between the housing 22 and the brake member 28. In one aspect or embodiment, as shown in FIG. 5, the first and second biasing members 24, 26 are received in first and second pockets 72, 74 defined by the brake member 28. The first and second pockets 72, 74 are defined by one or more protrusions 76 extending radially outward from the brake member 28. The first and second pockets 72, 74 may be a single annular pocket. The first and second biasing members 24, 26 may be one or more biasing members, as discussed in more detail below.

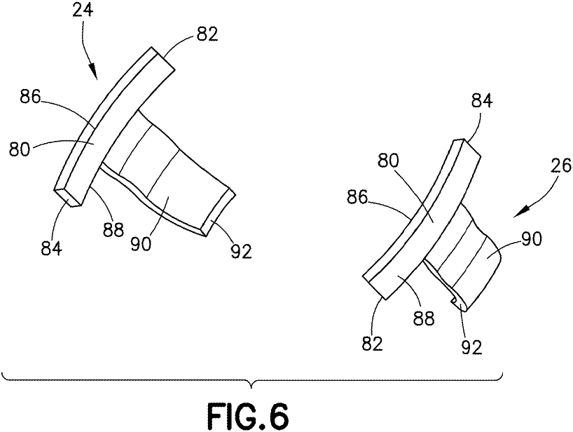

Referring to FIG. 6, the first and second biasing members 24, 26 each include a body 80 having a first end 82, a second end 84 positioned opposite the first end 82, a top surface 86, and a bottom surface 88 positioned opposite the top surface 86. Each biasing member 24, 26 also includes an extension portion 90 extending from the body 80 in a direction extending from the top surface 86 of the body 80 to the bottom surface 88 of the body 80. The extension portion 90 is resilient. The extension portion 90 may be resilient due to the geometry and/or material of the extension portion 90. The extension portion 90 extends from the bottom surface 88 of the body 80. The body 80 is arcuate, although other suitable shapes may be utilized. The arcuate shape of the body 80 of each biasing member 24, 26 is configured to conform to the outer surface of the brake member 28 and the inner surface of the housing 22. The extension portion 90 is wavy, although other suitable configurations may be utilized. The body 80 defines a width extending from the first end 82 of the body 80 to the second end 84 of the body 80. The extension portion 90 has a width less than the width of the body 80, although other suitable dimensions may be utilized.

The biasing members 24, 26 are T-shaped, although other suitable shapes and configurations may be utilized. The extension portion 90 extends from the body 80 at an equidistant point between the first and second ends 82, 84 of the body 80, although other suitable configurations may be utilized. A distal end 92 of the extension portion 90 is non-coplanar with the body 80.

In one aspect or embodiment, each biasing member 24, 26 is manufactured or formed from a metallic material, such as spring steel, although other suitable materials may be utilized.

In one aspect or embodiment, the body 80 is integrally formed with the extension portion 90. Each biasing member 24, 26 is configured to be formed as a single, unitary piece.

Although the first and second biasing members 24, 26 are provided, a single biasing member may be utilized. In one aspect or embodiment, the single biasing member may be the same as the first and second biasing members 24, 26 shown in in FIG. 6 and described above or may be wider with the arcuate body 80 extending a larger number of degrees to ensure a consistent axial biasing force is applied between the brake member 28 and the cartridge 36.

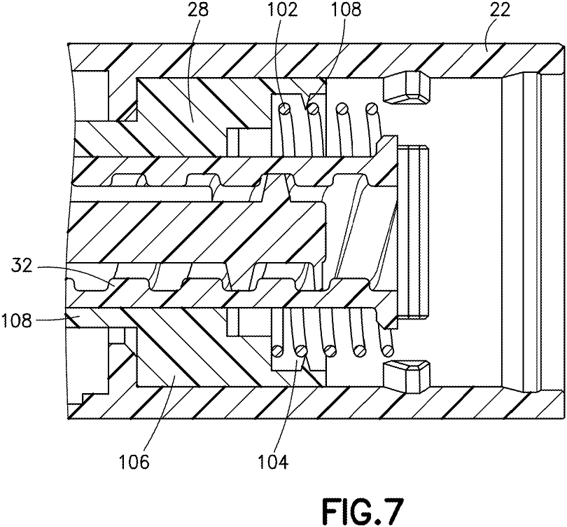

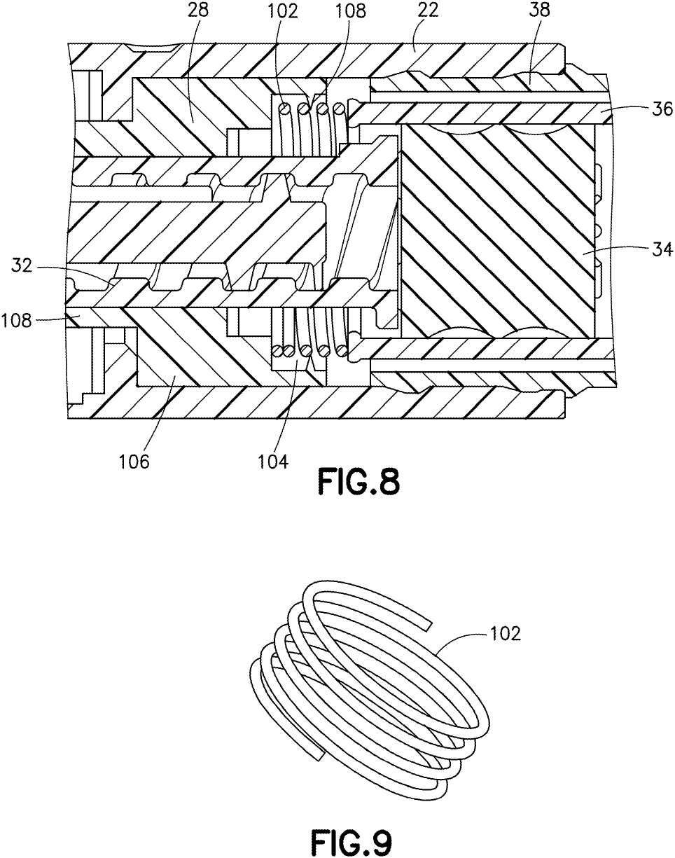

Referring to FIGS. 7-9, in a second aspect or embodiment of the present application, the biasing members 24, 26 are replaced within a single biasing member 102 formed as a compression spring. The biasing member 102 functions in the same manner as described above in connection with FIGS. 1-6. As shown in FIGS. 7 and 8, the biasing member 102 is received within a pocket 104 defined by the brake member 28. As shown in FIGS. 7 and 8, during assembly of the device 10 and prior to insertion of the cartridge 36 (FIG. 7), the biasing member 102 has an unbiased position where the biasing member 102 has a maximum length. After assembly of the device 10 with the cartridge 36 inserted and engaged with the biasing member 102 (FIG. 8), the biasing member 102 is in a biased position where the biasing member 102 is compressed with a length less than the maximum length of the biasing member 102.

The pocket 104 is defined by a shoulder portion 106 of the brake member 28, with the shoulder portion 106 extending radially outward from a body 108 of the brake member 28. The shoulder portion 106 includes a protrusion 110 extending radially inward into the pocket 104 with the protrusion 110 engaging a portion of the biasing member 102 to retain the biasing member 102 within the pocket 104.

Although the devices have been described in detail for the purpose of illustration based on what is currently considered to be the most practical and preferred embodiments, it is to be understood that such detail is solely for that purpose and that the systems and methods are not limited to the disclosed embodiments, but on the contrary, is intended to cover modifications and equivalent arrangements that are within the spirit and scope of the appended claims. For example, it is to be understood that the present systems and methods contemplate that, to the extent possible, one or more features of any embodiment can be combined with one or more features of any other embodiment.

The invention claimed is:

1. A biasing member for a medical injection device having a housing, a brake member, a cartridge, and an injection needle, the biasing member comprising:
a body having a first end, a second end positioned opposite the first end, a top surface, and a bottom surface positioned opposite the top surface; and
an extension portion extending from the body in a direction extending from the top surface of the body to the bottom surface of the body, wherein the extension portion is resilient.

2. The biasing member of claim 1, wherein the extension portion extends from the bottom surface of the body.

3. The biasing member of claim 1, wherein the body is arcuate.

4. The biasing member of claim 1, wherein the extension portion is wavy.

5. The biasing member of claim 1, wherein the body defines a width extending from the first end of the body to the second end of the body, and wherein the extension portion has a width less than the width of the body.

6. The biasing member of claim 1, wherein the biasing member is T-shaped.

7. The biasing member of claim 1, wherein the extension portion extends from the body at an equidistant point between the first and second ends of the body.

8. The biasing member of claim 1, wherein a distal end of the extension portion is non-coplanar with the body.

9. The biasing member of claim 1, wherein the biasing member comprises a metallic material.

10. The biasing member of claim 1, wherein the body is integrally formed with the extension portion.

11. The biasing member of claim 1, wherein the body and the extension portion are equal in thickness.

12. A medical injection device comprising:
a housing having a distal end and a proximal end;
a brake member received within the housing;
a cartridge received within the housing, the cartridge configured to receive a medicament;
a cartridge housing received within the housing distally of the brake member and configured to hold the cartridge;
a biasing member comprising a body having a first end, a second end positioned opposite the first end, a top surface, and a bottom surface positioned opposite the top surface; and an extension portion extending from the body in a direction extending from the top surface of the body to the bottom surface of the body, wherein the extension portion is resilient;
the biasing member received within the housing and positioned between the brake member and the cartridge;
an injection needle at the distal end of the housing and in fluid communication with the cartridge; and
an actuation member configured to actuate the medical injection device to deliver the composition through the injection needle.

13. The medical injection device of claim 12, wherein the biasing member is received within a pocket defined by the brake member.

14. The medical injection device of claim 13, wherein the pocket is defined by a radially extending protrusion of the brake member.

15. The medical injection device of claim 12, wherein the biasing member comprises first and second biasing members.

16. A medical injection device comprising:
a housing having a distal end and a proximal end;
a brake member received within the housing;
a cartridge received within the housing, the cartridge configured to receive a medicament;
a cartridge housing received within the housing distally of the brake member and configured to hold the cartridge;
a biasing member received within the housing and positioned between the brake member and the cartridge, the biasing member comprising a compression spring;
an injection needle at the distal end of the housing and in fluid communication with the cartridge; and
an actuation member configured to actuate the medical injection device to deliver the composition through the injection needle.

17. The medical injection device of claim 16, wherein the biasing member is received within a pocket defined by the brake member.

18. The medical injection device of claim 17, wherein the pocket is defined by a shoulder portion of the brake member, the shoulder portion extending radially outward from a body of the brake member.

19. The medical injection device of claim 18, wherein the shoulder portion comprises a protrusion extending radially inward into the pocket, the protrusion engaging a portion of the biasing member to retain the biasing member within the pocket.
